(19) United States
(12) Patent Application Publication
(10) Pub. No.: US 2016/0317454 A1
(43) Pub. Date: Nov. 3, 2016

(54) CONTROLLED RELEASE PHARMACEUTICAL COMPOSITION AND METHOD FOR MANUFACTURING THE SAME

(71) Applicant: Medical And Pharmaceutical Industry Technology And Development Center, New Taipei City (TW)

(72) Inventors: Chih-Chiang YANG, New Taipei City (TW); Yuan-Chih LE, New Taipei City (TW); Yu-Liang LIN, New Taipei City (TW); Yi-Ming LIOU, New Taipei City (TW)

(21) Appl. No.: 14/826,328

(22) Filed: Aug. 14, 2015

(30) Foreign Application Priority Data
Apr. 28, 2015 (TW) 104113463

(51) Int. Cl. A61K 9/50; A61K 9/48; A61K 31/403

(52) U.S. Cl. CPC A61K 9/5042; A61K 9/5026; A61K 31/403; A61K 9/5089; A61K 9/4866; A61K 9/4858

(57) ABSTRACT

A controlled release pharmaceutical composition is disclosed, which comprises: a drug core comprising 10-50 mg of Carvedilol or a pharmaceutical acceptable salt thereof, and a first pharmaceutical acceptable polymer, wherein a content of the first pharmaceutical acceptable polymer is 0.01-50% based on a total weight of the drug core; and a controlled release coating layer covering the drug core and comprising a second pharmaceutical acceptable polymer. In addition, a method for manufacturing the aforementioned controlled release pharmaceutical composition is also disclosed.

### CROSS REFERENCE TO RELATED APPLICATION

This application claims the benefits of the Taiwan Patent Application Serial Number 104113463, filed on Apr. 28, 2015, the subject matter of which is incorporated herein by reference.

### BACKGROUND OF THE INVENTION

1. Field of the Invention

The present invention relates to a controlled release pharmaceutical composition and a method for manufacturing the same and, more particularly, to a controlled release pharmaceutical composition capable of releasing Carvedilol in two-steps and a method for manufacturing the same.

2. Description of Related Art

According to investigation, the cardiovascular disease is one of the top ten leading causes of death, even though it is not the first among them. In Taiwan, ten thousand people suffer from the cardiovascular disease per year, and the population having cardiovascular disease in western countries is also increasing. Hence, the cardiovascular disease is a severe health threatening disease, and cannot be ignored. There are many kinds of cardiovascular disease, and the well-known cardiovascular disease includes hypertension, angina pectoris and heart failure.

In all kinds of the known cardiovascular disease, the treatment thereof depends upon the symptom of each patient. Except for the surgery, medical treatment is also one means to cure or prevent the cardiovascular disease. Herein, one of the known drugs for treating the cardiovascular disease is β-blocker, such as Atenolol and Carvedilol.

Carvedilol is one common drug for treating primary hypertension. The oral formulation of Carvedilol used in clinic is an immediate-released formulation, and it has to be administered several times to achieve the purpose of long-term administration. However, it is not convenient for patients to take this formulation several times a day.

Therefore, it is desirable to provide a novel formulation of Carvedilol, to achieve the purpose of long-term releasing with one single formulation, and further improve the administration convenience for the patient.

### SUMMARY OF THE INVENTION

The object of the present invention is to provide a controlled release pharmaceutical composition and a method for manufacturing the same, wherein the active ingredient Carvedilol in the controlled release pharmaceutical composition can be released in two steps, to further improve the effect of the active ingredient Carvedilol for treating cardiovascular diseases.

The controlled release pharmaceutical composition of the present invention comprises: a drug core comprising 10-50 mg of Carvedilol or a pharmaceutical acceptable salt thereof, and a first pharmaceutical acceptable polymer, wherein a content of the first pharmaceutical acceptable polymer is 0.01-50% based on a total weight of the drug core; and a controlled release coating layer covering the drug core and comprising a second pharmaceutical acceptable polymer.

In addition, the present invention further provides a method for manufacturing a controlled release pharmaceutical composition, which comprises the following steps: providing a drug core comprising 10-50 mg of Carvedilol or a pharmaceutical acceptable salt thereof, and a first pharmaceutical acceptable polymer, wherein a content of the first pharmaceutical acceptable polymer is 0.01-50% based on a total weight of the drug core; and forming a controlled release coating layer, which covers the drug core, to obtain a controlled release pharmaceutical composition, wherein the controlled release coating layer comprises a second pharmaceutical acceptable polymer.

In the controlled release pharmaceutical composition and the method for manufacturing the same of the present invention, a drug core comprising the Carvedilol or the pharmaceutical acceptable salt thereof is coated with a controlled release coating layer to form a long-term released particle. Compared with the commercial available controlled release formulation of Carvedilol that is a sustained release matrix formulation or a formulation using ester-based derivatives and hydrophilic polymers, the controlled release pharmaceutical composition of the present invention can achieve the purpose of two-steps releasing with one single formulation. Hence, the present invention provides another long-term released formulation for simple, infrequent daily administration.

In the method for manufacturing the controlled release pharmaceutical composition of the present invention, the drug core can be prepared by the following steps: providing an inert core; and coating the inert core with the Carvedilol or the pharmaceutical acceptable salt thereof, and the first pharmaceutical acceptable polymer at a predetermined temperature, to obtain an active ingredient layer covering the inert core. Herein, the predetermined temperature can be 30-60° C.; and preferably is 35-45° C. In this aspect, the obtained controlled release pharmaceutical composition of the present invention may comprise: an inert core and an active ingredient layer covering the inert core, wherein the active ingredient layer comprises: the Carvedilol or the pharmaceutical acceptable salt thereof, and the first pharmaceutical acceptable polymer.

In addition, in the method for manufacturing the controlled release pharmaceutical composition of the present invention, the controlled release coating layer can be formed at 15-40° C.; and preferably is formed at 15-25° C.

In the controlled release pharmaceutical composition and the method for manufacturing the same of the present invention, the inert core may comprise: at least one selected from the group consisting of sugar and microcrystalline cellulose. Preferably, the inert core is made of microcrystalline cellulose.

In the controlled release pharmaceutical composition and the method for manufacturing the same of the present invention, the active ingredient layer may further comprise: at least one selected from the group consisting of a binder, an anti-adhesive agent, a dispersant and a lubricant. Herein, specific examples of the lubricant comprises: at least one selected from the group consisting of corn starch, glyceryl monostearate, talc and metallic stearates; and preferably, the lubricant is talc. Herein, in the case that the binder, the anti-adhesive agent, the dispersant and/or the lubricant is used, the content thereof is not particularly limited; and can be adjusted according to the desirable released period and contents of other components.

In the controlled release pharmaceutical composition and the method for manufacturing the same of the present invention, the first pharmaceutical acceptable polymer may comprise: at least one selected from the group consisting of polyvinyl pyrrolidone, a copolymer of vinyl pyrrolidone and vinyl alcohol, a copolymer of polyvinyl pyrrolidone and ethenyl acetate, a copolymer of polyvinyl pyrrolidone and vinyl chloride, a copolymer of polyvinyl pyrrolidone and vinyl butyrate, a copolymer of polyvinyl pyrrolidone and vinyl laurate, a copolymer of polyvinyl pyrrolidone and vinyl stearate, hydroxypropyl cellulose, hydroxypropyl alkyl cellulose such as hydroxypropyl methyl cellulose, poly ethylene oxide, sodium carboxymethyl cellulose, dextran, gum acacia, starch and gelatin. Preferably, the first pharmaceutical acceptable polymer is hydroxypropyl methyl cellulose. Furthermore, a content of the first pharmaceutical acceptable polymer can be 0.01-50%; preferably is 0.1-30%; and more preferably is 1-15%, based on a total weight of the drug core.

In the controlled release pharmaceutical composition and the method for manufacturing the same of the present invention, the second pharmaceutical acceptable polymer can comprise: at least one selected from the group consisting of a copolymer of methacrylate and methacrylate having an amino groups, a copolymer of methylpropenoic acid and acrylate, a copolymer of methylpropenoic acid and methacrylate, a copolymer of acrylate and methacrylate having an amino groups, polyacrylate, a copolymer of methylpropenoic acid, acrylate and methacrylate, poly(methacrylate), a copolymer of maleic anhydride and vinyl methyl ether, shellac, ethyl cellulose, cellulose acetate phthalate, hydroxypropyl methylcellulose phthalate, hydroxypropyl methylcellulose acetate succinate, polyvinyl acetate phthalate, a copolymer of ethyl acrylate and methyl methacrylate, and a copolymer of methylpropenoic acid and ethyl acrylate. Preferably, the second pharmaceutical acceptable polymer comprises: at least one selected from the group consisting of ethyl cellulose, a copolymer of ethyl acrylate and methyl methacrylate, and a copolymer of methylpropenoic acid and ethyl acrylate. In addition, a content of the second pharmaceutical acceptable polymer can be 50-100% based on a total weight of the controlled release coating layer.

Furthermore, in the controlled release pharmaceutical composition and the method for manufacturing the same of the present invention, the controlled release coating layer may selectively further comprise: at least one selected from the group consisting of a plasticizer, a slip agent and a porogen. Herein, specific examples of the plasticizer can comprise at least one selected from the group consisting of glyceroltriacetate, tributyl citrate, triethyl citrate, acetyl tributyl citrate, diethyl phthalate, dibutyl sebacate, polyethylene glycol and polypropylene glycol. Preferably, the plasticizer is triethyl citrate. In addition, in the case that the plasticizer, the slip agent and/or the porogen is used, the content thereof is not particularly limited; and can be adjusted according to the desirable released period and contents of other components. Preferably, when the controlled release coating layer further comprises a plasticizer, a content of the plasticizer is 0.5-30% based on a total weight of the controlled release coating layer.

Other objects, advantages, and novel features of the invention will become more apparent from the following detailed description when taken in conjunction with the accompanying drawings.

### BRIEF DESCRIPTION OF THE DRAWINGS

FIG. 1 is a schematic view showing a process for manufacturing a controlled release pharmaceutical composition of the present invention.

FIG. 2 is a dissolution curve of a controlled release pharmaceutical composition of Embodiment 3 of the present invention.

FIG. 3 is a dissolution curve of a controlled release pharmaceutical composition of Embodiment 4 of the present invention.

FIG. 4 is a dissolution curve of a controlled release pharmaceutical composition of Embodiment 5 of the present invention.

FIG. 5 is a dissolution curve of a controlled release pharmaceutical composition of Embodiment 6 of the present invention.

FIG. 6 is a dissolution curve of a controlled release pharmaceutical composition of Embodiment 7 of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

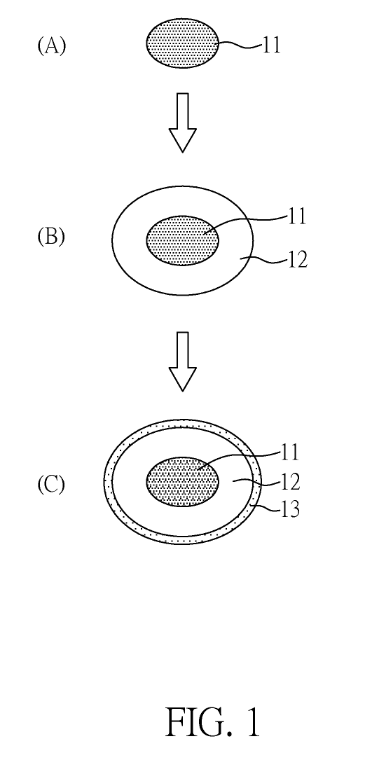

In the following embodiments of the present invention, the process for manufacturing controlled release pharmaceutical compositions is represented by FIG. 1. First, as shown in FIG. 1(A), an inert core 11 is provided. Next, as shown in FIG. 1(B), an active ingredient (Carvedilol or a pharmaceutical acceptable salt thereof) and a first pharmaceutical acceptable polymer, sometimes selectively co-used with one or several excipients such as a binder, an anti-adhesive agent, a dispersant and a lubricant, are mixed in different ratios to form a coating solution. The inert core 11 is coated with the coating solution through a fluidized bed coating, and an active ingredient layer 12 covering the inert core 11 is formed. After the aforementioned process, a drug core is obtained. Then, as shown in FIG. 1(C), a second pharmaceutical acceptable polymer, sometimes selectively co-used with one or several excipients such as a plasticizer, a slip agent and a porogen, are applied on the drug core through a film coating process to form a controlled release coating layer 13. Thus, the controlled release pharmaceutical composition of the present invention is obtained. The controlled release pharmaceutical composition can also be formulated into a capsule or a tablet, if it is necessary.

After the aforementioned process, as shown in FIG. 1(C), the controlled release pharmaceutical composition of the present invention comprises: a drug core comprising an inert core 11 and an active ingredient layer 12 covering the inert core 11, wherein the active ingredient layer 12 comprises Carvedilol or a pharmaceutical acceptable salt thereof, and a first pharmaceutical acceptable polymer; and a controlled release coating layer 13 covering the drug core and comprising a second pharmaceutical acceptable polymer.

In the following embodiments of the present invention, the active ingredient, Carvedilol phosphate (hereinafter, named as “Carvedilol”), is available from Polpharma SA Pharmaceutical Works, Poland; microcrystalline cellulose is available from Pharmatrans Sanaq AG, Switzerland; a copolymer of ethyl acrylate and methyl methacrylate (Eudragit NE30D) and a copolymer of methylpropenoic acid and ethyl acrylate (Eudragit L30D-55) are available from Evonik Industries AG, Germany; and hydrogen chloride and potassium phosphate monobasic are available from Riedel-de-Haen, USA. Other chemicals are also commercially available, and are chemical grade reagents.

### Embodiment 1

TABLE 1
Components for the drug core

| Component | % |
|---|---|
| Carvedilol | 19.12 |
| Microcrystalline cellulose | 76.48 |
| Talc | 1.34 |
| hydroxypropyl methyl cellulose | 3.06 |

TABLE 2
Components for the controlled release coating layer

| Component | % |
|---|---|
| Ethyl cellulose | 100 |

The controlled release pharmaceutical composition of the present embodiment is formulated with the components indicated in Tables 1 and 2. First, Carvedilol and talc were dispersed in a hydroxypropyl methyl cellulose polymer solution to form a coating solution. An inert core made of microcrystalline cellulose was placed in a fluidized bed coating machine and heated to 40° C., followed by uniformly spraying the coating solution to coat the inert core with the sprayed coating solution to form a drug core. Then, a coating solution made of Ethyl cellulose was uniformly sprayed to coat the drug core with the sprayed coating solution in the fluidized bed coating machine at 40° C., to obtain the controlled release particle of the present embodiment.

### Embodiment 2

TABLE 3
Components for the drug core

| Component | % |
|---|---|
| Carvedilol | 19.12 |
| Microcrystalline cellulose | 76.48 |
| Talc | 1.34 |
| hydroxypropyl methyl cellulose | 3.06 |

TABLE 4
Components for the controlled release coating layer

| Component | % |
|---|---|
| Eudragit NE30D | 92.6 |
| (a copolymer of ethyl acrylate and methyl methacrylate) | |
| PlasAcryl T20 (containing triethyl citrate) | 7.4 |

The controlled release pharmaceutical composition of the present embodiment is formulated with the components indicated in Tables 3 and 4. First, Carvedilol and talc were dispersed in a hydroxypropyl methyl cellulose polymer solution to form a coating solution. An inert core made of microcrystalline cellulose was placed in a fluidized bed coating machine and heated to 40° C., followed by uniformly spraying the coating solution to coat the inert core with the sprayed coating solution to form a drug core. Then, a coating solution consisting of Eudragit NE30D and PlasAcryl T20 was uniformly sprayed to coat the drug core with the sprayed coating solution in the fluidized bed coating machine at 20° C., to obtain the controlled release particle of the present embodiment.

### Embodiment 3

TABLE 5
Components for the drug core

| Component | % |
|---|---|
| Carvedilol | 19.12 |
| Microcrystalline cellulose | 76.48 |
| Talc | 1.34 |
| hydroxypropyl methyl cellulose | 3.06 |

TABLE 6
Components for the controlled release coating layer

| Component | % |
|---|---|
| Eudragit NE30D | 35.7 |
| (a copolymer of ethyl acrylate and methyl methacrylate) | |
| Eudragit L30D-55 | 53.6 |
| (a copolymer of methylpropenoic acid and ethyl acrylate) | |
| PlasAcryl T20 (containing triethyl citrate) | 10.7 |

The controlled release pharmaceutical composition of the present embodiment is formulated with the components indicated in Tables 5 and 6. First, Carvedilol and talc were dispersed in a hydroxypropyl methyl cellulose polymer solution to form a coating solution. An inert core made of microcrystalline cellulose was placed in a fluidized bed coating machine and heated to 40° C., followed by uniformly spraying the coating solution to coat the inert core with the sprayed coating solution to form a drug core. Then, a coating solution consisting of Eudragit NE30D, Eudragit L30D-55 and PlasAcryl T20 was uniformly sprayed to coat the drug core with the sprayed coating solution in the fluidized bed coating machine at 20° C., to obtain the controlled release particle of the present embodiment.

### Embodiment 4

TABLE 7
Components for the drug core

| Component | % |
|---|---|
| Carvedilol | 10.99 |
| Microcrystalline cellulose | 85.93 |
| Talc | 0.76 |
| hydroxypropyl methyl cellulose | 1.76 |
| polysorbate | 0.56 |

TABLE 8
Components for the controlled release coating layer

| Component | % |
|---|---|
| Eudragit L30D-55 | 66.7 |
| (a copolymer of methylpropenoic acid and ethyl acrylate) | |
| PlasAcryl T20 (containing triethyl citrate) | 13.3 |
| Mannitol | 20.0 |

The controlled release pharmaceutical composition of the present embodiment is formulated with the components indicated in Tables 7 and 8. First, Carvedilol and talc were dispersed in a polysorbate solution containing hydroxypropyl methyl cellulose polymer to form a coating solution. An inert core made of microcrystalline cellulose was placed in a fluidized bed coating machine and heated to 39° C., followed by uniformly spraying the coating solution to coat the inert core with the sprayed coating solution to form a drug core. Then, a coating solution consisting of Eudragit L30D-55, PlasAcryl T20 and mannitol was uniformly sprayed to coat the drug core with the sprayed coating solution in the fluidized bed coating machine at 20° C., to obtain the controlled release particle of the present embodiment.

### Embodiment 5

TABLE 9
Components for the drug core

| Component | % |
|---|---|
| Carvedilol | 10.99 |
| Microcrystalline cellulose | 86.47 |
| Talc | 0.77 |
| hydroxypropyl methyl cellulose | 1.77 |

TABLE 10
Components for the controlled release coating layer

| Component | % |
|---|---|
| Eudragit NE30D | 21.1 |
| (a copolymer of ethyl acrylate and methyl methacrylate) | |
| Eudragit L30D-55 | 49.1 |
| (a copolymer of methylpropenoic acid and ethyl acrylate) | |
| PlasAcryl T20 (containing triethyl citrate) | 9.8 |
| Mannitol | 20.0 |

The controlled release pharmaceutical composition of the present embodiment is formulated with the components indicated in Tables 9 and 10. First, Carvedilol and talc were dispersed in a hydroxypropyl methyl cellulose polymer solution to form a coating solution. An inert core made of microcrystalline cellulose was placed in a fluidized bed coating machine and heated to 40° C., followed by uniformly spraying the coating solution to coat the inert core with the sprayed coating solution to form a drug core. Then, a coating solution consisting of Eudragit NE30D, Eudragit L30D-55, PlasAcryl T20 and mannitol was uniformly sprayed to coat the drug core with the sprayed coating solution in the fluidized bed coating machine at 20° C., to obtain the controlled release particle of the present embodiment.

### Embodiment 6

TABLE 11
Components for the drug core

| Component | % |
|---|---|
| Carvedilol | 19.17 |
| Microcrystalline cellulose | 75.38 |
| Talc | 1.34 |
| hydroxypropyl methyl cellulose | 3.12 |
| polysorbate | 0.99 |

TABLE 12
Components for the controlled release coating layer

| Component | % |
|---|---|
| Eudragit L30D-55 | 66.7 |
| (a copolymer of methylpropenoic acid and ethyl acrylate) | |
| PlasAcryl T20 (containing triethyl citrate) | 13.3 |
| Mannitol | 20.0 |

The controlled release pharmaceutical composition of the present embodiment is formulated with the components indicated in Tables 11 and 12. First, Carvedilol and talc were dispersed in a polysorbate solution containing hydroxypropyl methyl cellulose polymer to form a coating solution. An inert core made of microcrystalline cellulose was placed in a fluidized bed coating machine and heated to 38° C., followed by uniformly spraying the coating solution to coat the inert core with the sprayed coating solution to form a drug core. Then, a coating solution consisting of Eudragit L30D-55, PlasAcryl T20 and mannitol was uniformly sprayed to coat the drug core with the sprayed coating solution in the fluidized bed coating machine at 20° C., to obtain the controlled release particle of the present embodiment.

### Embodiment 7

TABLE 13
Components for the drug core

| Component | % |
|---|---|
| Carvedilol | 19.26 |
| Microcrystalline cellulose | 75.33 |
| Talc | 1.33 |
| hydroxypropyl methyl cellulose | 3.08 |
| polysorbate | 0.99 |

TABLE 14
Components for the controlled release coating layer

| Component | % |
|---|---|
| Eudragit L30D-55 | 83.3 |
| (a copolymer of methylpropenoic acid and ethyl acrylate) | |
| PlasAcryl T20 (containing triethyl citrate) | 16.7 |

The controlled release pharmaceutical composition of the present embodiment is formulated with the components indicated in Tables 13 and 14. First, Carvedilol and talc were dispersed in a polysorbate solution containing hydroxypropyl methyl cellulose polymer to form a coating solution. An inert core made of microcrystalline cellulose was placed in a fluidized bed coating machine and heated to 42° C., followed by uniformly spraying the coating solution to coat the inert core with the sprayed coating solution to form a drug core. Then, a coating solution consisting of Eudragit L30D-55 and PlasAcryl T20 was uniformly sprayed to coat the drug core with the sprayed coating solution in the fluidized bed coating machine at 20° C., to obtain the controlled release particle of the present embodiment.

### Test Example

Hereinafter, the obtained controlled release pharmaceutical compositions prepared in Embodiments 3-7 were used to perform the dissolution test, which was performed according to USP34-NF29 General Chapter <711> Dissolution. Herein, the dissolution test was performed with Apparatus 2 (Paddle) at 37° C. During the begining 2 hours, 0.1 N HCl was used as a solvent; and after 2 hours passed, a phosphate buffer solution was added therein to change the pH value of the solvent into 6.8. The solvent volume was 900 ml. The solvent was stirred at 100 rpm/min. The dissolution test was performed for 24 hours. The solvent was sampled at suitable time points, and analyzed with a high pressure liquid chomotagraphy (HPLC) to obtain the dissolution rate of Carvedilol. The HPLC (Agilent 1100, USA) was performed with Inertsil C8 (5 μm, 4.6×150 mm) to examine the concentration of Carvedilol in the samples. The mobile phase was acetonitrile/phosphate buffer solution (31:69, pH 2.0), the retention time was 7 min, and the detecting wavelength was 240 nm.

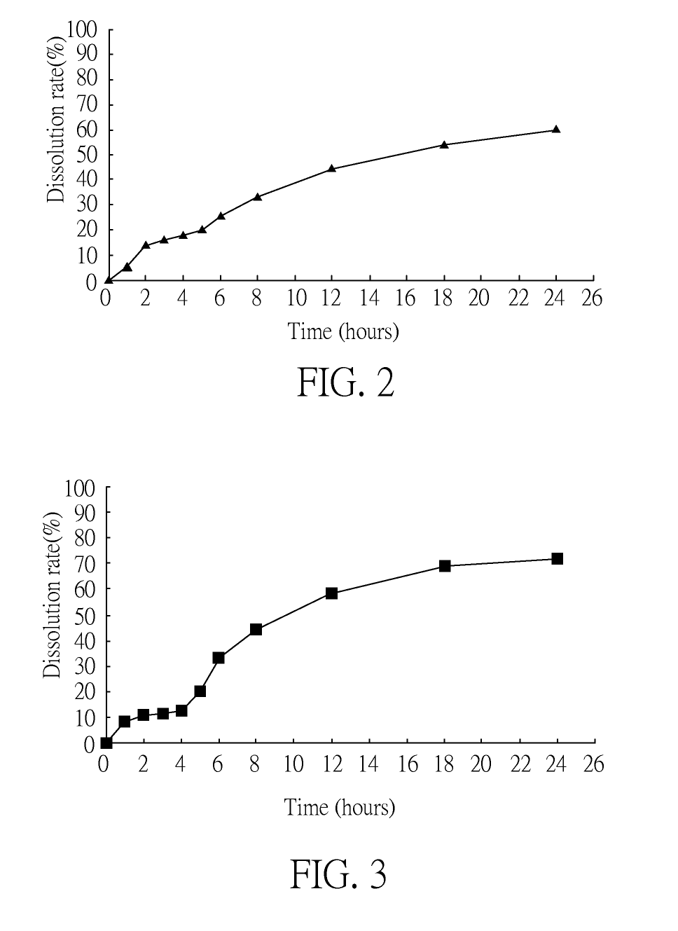

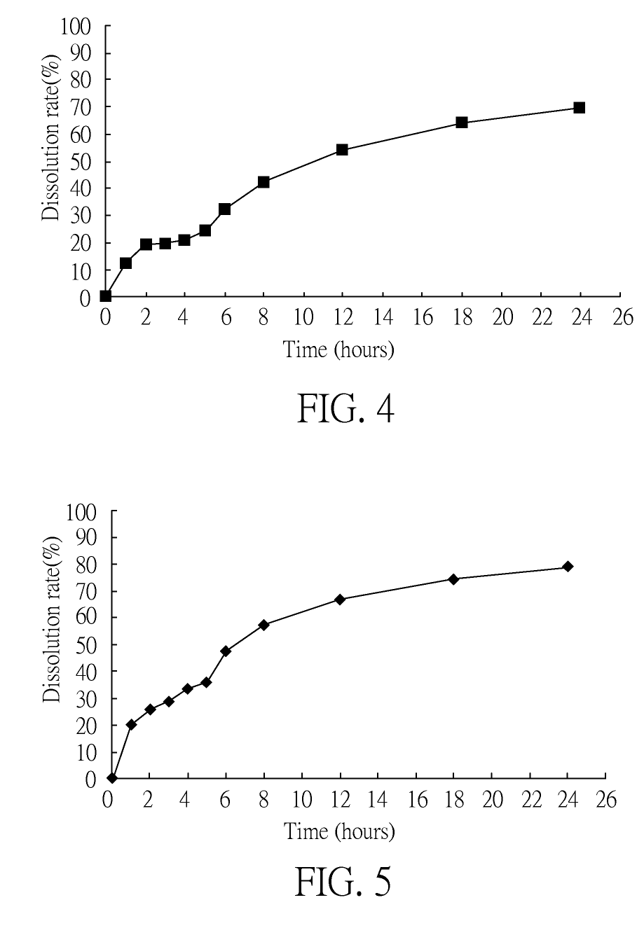

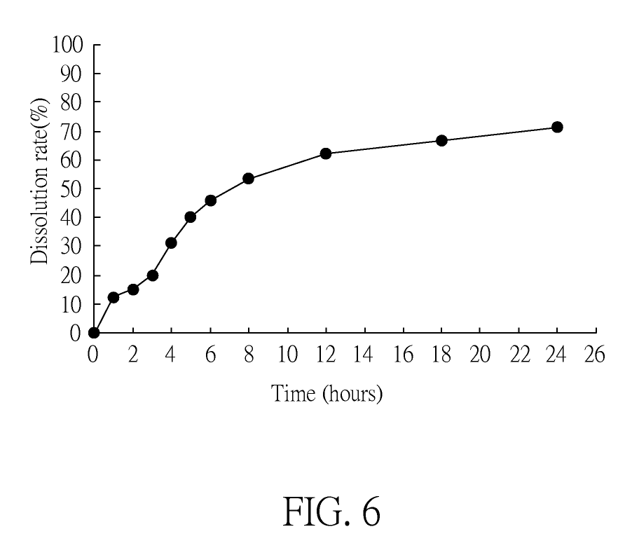

FIGS. 2-6 are dissolution curves of controlled release pharmaceutical compositions of Embodiments 3-7 of the present invention. The results indicate that the releasing time of the controlled release pharmaceutical compositions of the present invention can be extended to at least 24 hours. More specifically, the present test example was performed to mimic the change of the controlled release pharmaceutical compositions of the present invention in the gastrointestinal condition; and the results indicate that the releasing time of the controlled release pharmaceutical compositions in this condition can be extended to at least 24 hours. Especially, in the case using the controlled release pharmaceutical compositions of Embodiments 3 and 5 in which the controlled release coating layer contains Eudragit NE30D (a copolymer of ethyl acrylate and methyl methacrylate) and Eudragit L30D-55 (a copolymer of methylpropenoic acid and ethyl acrylate), when the pH is less than 5.5, the active ingredient can be slowly released via the polymer network of Eudragit NE30D; and when the pH is 6.8, Eudragit L30D-55 is degraded and the active ingredient contained in the drug core can be dissolved. Hence, when the controlled release pharmaceutical composition of the present invention is orally administered, the active ingredient can be slowly released; and thus the purpose of simple, infrequent daily administration can be achieved.

Although the present invention has been explained in relation to its preferred embodiment, it is to be understood that many other possible modifications and variations can be made without departing from the spirit and scope of the invention as hereinafter claimed.

1. A controlled release pharmaceutical composition, comprising:
a drug core comprising an inert core and an active ingredient layer covering the inert core, wherein the active ingredient layer comprises: 10-50 mg of Carvedilol or a pharmaceutical acceptable salt thereof, and a first pharmaceutical acceptable polymer, wherein a content of the first pharmaceutical acceptable polymer is 0.01-50% based on a weight of the drug core; and
a controlled release coating layer covering the drug core and comprising a second pharmaceutical acceptable polymer, wherein the second pharmaceutical acceptable polymer a copolymer of ethyl acrylate and methyl methacrylate, a copolymer of methylpropenoic acid and ethyl acrylate, or a combination thereof.

2. (canceled)

3. The controlled release pharmaceutical composition of claim 1, wherein the inert core comprises: at least one selected from the group consisting of sugar and microcrystalline cellulose.

4. The controlled release pharmaceutical composition of claim 1, wherein the active ingredient layer further comprises: at least one selected from the group consisting of a binder, an anti-adhesive agent, a dispersant and a lubricant.

5. The controlled release pharmaceutical composition of claim 4, wherein the lubricant comprises: at least one selected from the group consisting of corn starch, glyceryl monostearate, talc and metallic stearates.

6. The controlled release pharmaceutical composition of claim 1, wherein the first pharmaceutical acceptable polymer comprises: at least one selected from the group consisting of polyvinyl pyrrolidone, a copolymer of vinyl pyrrolidone and vinyl alcohol, a copolymer of polyvinyl pyrrolidone and ethenyl acetate, a copolymer of polyvinyl and vinyl chloride, a copolymer of polyvinyl pyrrolidone and vinyl butyrate, a copolymer of polyvinyl pyrrolidone and vinyl laurate, a copolymer of polyvinyl pyrrolidone and vinyl stearate, hydroxypropyl cellulose, hydroxypropyl alkyl cellulose, poly ethylene oxide, carboxymethyl cellulose, dextran, gum acacia, starch and gelatin.

7-8. (canceled)

9. The controlled release pharmaceutical composition of claim 1, wherein the controlled release coating layer further comprises: at least one selected from the group consisting of a plasticizer, a slip agent and a porogen.

10. The controlled release pharmaceutical composition of claim 9, wherein the plasticizer comprises at least one selected from the group consisting of glyceroltriacetate, tributyl citrate, triethyl citrate, acetyl tributyl citrate, diethyl phthalate, dibutyl sebacate, polyethylene glycol and polypropylene glycol.

11. The controlled release pharmaceutical composition of claim 1, wherein a content of the second pharmaceutical acceptable polymer is 50-100% based on a total weight of the controlled release coating layer.

12. The controlled release pharmaceutical composition of claim 1, wherein when the controlled release coating layer further comprises a plasticizer, a content of the plasticizer is 0.5-30% based on a total weight of the controlled release coating layer.

13. A method for manufacturing a controlled release pharmaceutical composition, comprising the following steps:
providing an inert core;
coating the inert core with Carvedilol or a pharmaceutical acceptable salt thereof, and a first pharmaceutical acceptable polymer, at a predetermined temperature to obtain an active ingredient layer covering the inert core, wherein the active ingredient layer comprises 10-50 mg of the Carvedilol or pharmaceutical acceptable salt thereof, and the first pharmaceutical acceptable polymer, and wherein a content of the first pharmaceutical acceptable polymer is 0.01-50% based on a total weight of the drug core; and
forming a controlled release coating layer, which covers the drug core, to obtain a controlled release pharmaceutical composition, wherein the controlled release coating layer comprises a second pharmaceutical acceptable polymer, and wherein the second pharmaceutical acceptable polymer is a copolymer of ethyl acrylate and methyl methacrylate, a copolymer of methylpropenoic acid and ethyl acrylate, or a combination thereof.

14. (canceled)

15. The method of claim 13, wherein the predetermined temperature is 30-60° C.

16. The method of claim 13, wherein the controlled release coating layer is formed at 15-40° C.

17. The method of claim 13, wherein the inert core comprises: at least one selected from the group consisting of sugar and microcrystalline cellulose.

18. The method of claim 13, wherein a content of the second pharmaceutical acceptable polymer is 50-100% based on a total weight of the controlled release coating layer.

19. The method of claim 13, wherein the controlled release coating layer further comprises: at least one selected from the group consisting of a plasticizer, a slip agent and a porogen.

20. The method of claim 19, wherein when the controlled release coating layer further comprises a plasticizer, a content of the plasticizer is 0.5-30% based on a total weight of the controlled release coating layer.
